(19) United States
(12) Patent Application Publication
(10) Pub. No.: US 2010/0048609 A1
(43) Pub. Date: Feb. 25, 2010

(54) PHARMACEUTICAL DOSAGE FORMS FOR (+)-1,4-DIHYDRO-7-[(3S,4S)-3-METHOXY-4-(METHYLAMINO)-1-PYRROLIDINYL]-4-OXO-1-(2-THIAZOLYL)-1,8-NAPHTHYRIDINE-3-CARBOXYLIC ACID

(76) Inventor: Jeffrey W. Jacobs, San Mateo, CA (US)

Correspondence Address: JONES DAY, 222 EAST 41ST ST, NEW YORK, NY, 10017, US

(21) Appl. No.: 12/376,118

(22) PCT Filed: Aug. 1, 2007
(86) PCT No.: PCT/US07/17248

§ 371 Date: Nov. 9, 2009

Related U.S. Application Data
(60) Provisional application No. 60/835,148, filed on Aug. 1, 2006.

(51) Int. Cl. A61K 31/4375; A61P 35/00

(52) U.S. Cl. 514/300

(57) ABSTRACT

Provided herein are light protective pharmaceutical packages for enantiomerically pme (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid. In certain embodiments, the pharmaceutical packages comprise a glass vial containing SNS-595 drug product within a secondary container.

This application claims the benefit of priority of U.S. provisional application No. 60/835,148, filed Aug. 1, 2006, the contents of which are hereby incorporated by reference in their entirety.

### 1. FIELD OF THE INVENTION

Provided herein are light protective finished pharmaceutical dosage forms for enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid and formulations thereof. The pharmaceutical dosage forms provided herein are capable of protecting the compound or a drug product comprising the compound from exposure to light thereby providing a more stable and safer product for use in humans.

### 2. BACKGROUND OF THE INVENTION

(+)-1,4-Dihydro-7-[(3S,4S)-3-methoxy-4-(methylamino)-1-pyrrolidinyl]-4-oxo-1-(2-thiazolyl)-1,8-naphthyridine-3-carboxylic acid is known for its anti-tumor activity (see, Tsuzuki et al., 2004, J. Med. Chem., 47:2097-2106, and Tomita et al., 2002, J. Med. Chem., 45: 5564-5575). Treatment of the following cancers with (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid has been proposed in the literature: bladder cancer, breast cancer, cervical cancer, colon cancer, esophageal cancer, head and neck cancer, liver cancer, lung cancer, melanoma, myeloma, neuroblastoma, ovarian cancer, pancreatic cancer, prostate cancer, renal cancer, sarcoma, skin cancer, stomach cancer, testicular cancer, thyroid cancer and uterine cancer. Various dosing regimens for the use of this compound have been reported, for example, see, U.S. Patent Application Pub. Nos. 2005-0203120; 2005-0215583 and 2006-0025437, which are incorporated herein by reference in their entireties.

Since (+)-1,4-Dihydro-7-[(3S,4S)-3-methoxy-4-(methylamino)-1-pyrrolidinyl]-4-oxo-1-(2-thiazolyl)-1,8-naphthyridine-3-carboxylic acid is an injectable drug product that is used in cancer or other immuno-comprised patients, product stability is important.

### 3. SUMMARY

Provided herein are light protective finished pharmaceutical dosage forms for enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid. The finished pharmaceutical dosage forms are capable of protecting the compound or a drug product comprising the compound from exposure to light while providing and/or maintaining ease of access for the user and easy handling during shipping without loss of activity of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

In certain embodiments, the finished pharmaceutical dosage forms provided herein comprise a primary container, such as a glass or plastic vial or a syringe containing enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid alone or within a secondary container for the vial or the syringe. In certain embodiments, the secondary container is selected from an opaque foil-lined overwrap or pouch, a cardboard box, such as a white, or brown, or other color cardboard box and a corrugated box. The cardboard box or corrugated box may further comprise cardboard inserts or foam-lined inserts. In one embodiment, the secondary container is an opaque foil-lined pouch.

In certain embodiments, the finished pharmaceutical dosage forms provided herein allow less than about 6 lux penetration. In certain embodiments, the finished pharmaceutical dosage forms allow about 3.5 to about 5.5 lux penetration.

In one embodiment, the finished pharmaceutical dosage form comprises a glass vial containing a drug product, wherein the drug product comprises enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid and an acid, and a secondary container for the vial. In one embodiment, the drug product is for IV administration. In certain embodiments, the finished pharmaceutical dosage form comprises a foil-lined pouch containing the drug product for IV administration.

In one embodiment, the pharmaceutical package comprises a single light-protective container. For instance, in certain embodiments, the pharmaceutical package can comprise a light-protective vial. The single light-protective container can comprise any of the light protective materials described in the sections below.

The finished pharmaceutical dosage forms provided herein are useful for storing and shipping enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid or a drug product comprising the compound for extended periods of time. In certain embodiments, enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid stored in the finished pharmaceutical dosage forms is stable from about 1 month up to about 36 months or more.

Also provided are methods of making the finished pharmaceutical dosage forms. The foil-lined pouch provided herein can be in any design, shape or form, irregular or uniform. In certain embodiments, the pouch has a uniform shape such as square, rectangle, circles and oval in order to facilitate the sealing and manufacturing processes. The pouch can be formed in sequential steps of folding and sealing. Sealing can be accomplished by heat, ultrasound, laser, or adhesive or any other means known to one of skill in the art.

In another embodiment, provided herein is a finished pharmaceutical dosage form of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid which comprises about 1 mg to about 100 mg of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid in an aqueous solution, optionally containing an acid to adjust the pH of the solution to less than about 4. The finished pharmaceutical dosage form is protected from light of wavelength about 200 nm to about 700 arm. The light protection can be provided in a number of ways, including by use of the pharmaceutical packages described herein.

### 4. BRIEF DESCRIPTION OF DRAWINGS

FIG. 1. illustrates an exemplary clear glass vial for use in the finished pharmaceutical dosage forms for (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

FIG. 2. illustrates an exemplary plastic vial for use in the finished pharmaceutical dosage forms for (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

FIGS. 3 (A)-(C). illustrate an exemplary foil-lined pouches for use as a secondary container for a vial containing (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid drug product.

FIG. 4. provides an exemplary white cardboard box with a black foam liner for use as a secondary container for a vial containing (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

FIG. 5. provides an exemplary black foam liner used with a cardboard box as a secondary container for a vial containing (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

FIG. 6. provides an exemplary brown corrugated box for use as a secondary container for a vial containing (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

FIG. 7. illustrates an exemplary foil-lined IV bag containing the drug product for (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

### 5. DETAILED DESCRIPTION OF THE INVENTION

### 5.1 Definitions

As used herein, “SNS-595” refers to (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid. The compound is also known as AG-7352. The chemical structure of the compound is provided below. Unless otherwise designated, SNS-595 or (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid refers to the enantiomerically pure form of the compound.

As used herein, enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid is substantially free from (−)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid (i.e., in enantiomeric excess). In other words, the “(+)” form of 1,4-dihydro-7-[(3S,4S)-3-methoxy-4-(methylamino)-1-pyrrolidinyl]-4-oxo-1-(2-thiazolyl)-1,8-naphthyridine-3-carboxylic acid is substantially free from the “(−)” form of the compound and is, thus, in enantiomeric excess of the “(−)” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight or more than 97% by weight of the enantiomer.

As used herein, “SNS-595 drug product” refers to a formulation comprising SNS-595. In certain embodiments, the drug product comprises SNS-595 and an acid.

As used herein, “photostability of SNS-595 drug product” refers to the stability of the drug product to ambient light. The stability can be measured by methods known to one of skill in the art, including but not limited to visual observation of discoloration, precipitate formation or chromatographic methods. In certain embodiments, the stability of the drug product is measured by HPLC.

As used herein “photostable drug product” refers to the SNS-595 drug product containing less than about 2% total impurities associated with light exposure as measured by HPLC. In certain embodiments, the stable drug product contains less than about 1.5%, 1.4%, 1.3%, 1.2%, 1.1. %, 1%, 0.9%, 0.8%, 0.7%, 0.6%, 0.5%, 0.4%, 0.3%, 0.2% or 0.1% total impurities.

As used herein, “ambient light” refers to a light from 50-60 foot candles/500-700 lux. As known in the art, direct sunlight exposure for about an hour is equivalent to about 100,000 lux hours of white light. Table 1 in Section 4.3 provides intensity of white light exposure at various locations indoors and outdoors.

As used herein and unless otherwise indicated, the terms “treat,” “treating” and “treatment” refer to alleviating or reducing the severity of a disease or a symptom associated with the disease or condition being treated.

The term “prevention” includes the inhibition of a disease or disorder or a symptom of the particular disease or disorder. In some embodiments, patients with familial history of cancer are candidates for preventive regimens. Generally, the term “preventing” refers to administration of the drug prior to the onset of symptoms, particularly to patients at risk of cancer.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein “subject” is an animal, typically a mammal, including human, such as a patient.

As used herein, the term “cancer” includes, but is not limited to, solid tumors and blood born tumors. In certain embodiments, cancer is cancer of organs, blood or vessels, including, but not limited to, cancer of the bladder, bone or blood, brain, breast, cervix, chest, colon, endrometrium, esophagus, eye, head, kidney, liver, lung, mouth, neck, ovaries, pancreas, prostate, rectum, stomach, testis, throat or uterus. In certain embodiments, cancer is cancer of skin tissues. In certain embodiments, the cancer is a hematologic malignancy, such as a leukemia, lymphoma (Non-Hodgkin's Lymphoma), Hodgkin's disease (also called Hodgkin's Lymphoma) or myeloma. In certain embodiments, the leukemia is chronic lymphocytic leukemia, chronic myeloid leukemia, acute lymphocytic leukemia, acute myelogenous leukemia or acute myeloblastic leukemia. In certain embodiments, the cancer comprises solid tumor. In certain embodiments, the cancer can be relapsed, refractory or resistant to conventional therapy. In certain embodiments, the cancer can be metastatic.

The term “relapsed” refers to a return of cancer cells or symptoms in patients who have had a previous remission of cancer after therapy.

The term “refractory or resistant” refers to patients that, even after treatment or intensive treatment, have residual cancer cells in their body.

The term “gauge” or “ga” as used herein is a unit measuring 1/100,000 inch, or 0.00001 inch.

The term “mil” as used herein is a unit measuring 1/1,000 inch or 0.001 inch.

The term “basis weight” when used herein in reference to paper is defined as the weight of the paper in pounds that would be required to cover an area of 3,000 square feet (sf).

The term “coating weight” when used herein in reference to plastic is the weight of the plastic in pounds that would be required to cover an area of 3,000 square feet at a thickness of 1 mil.

### 5.2 SNS-595 Drug Product

SNS-595 refers to enantiomerically pure (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid and has the following chemical structure:

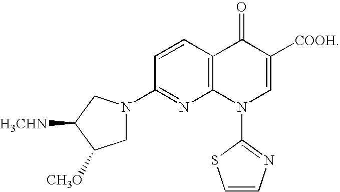

SNS-595 can be prepared by methods known to one of skill in the art, for example, according to the preparation procedure for Example C-1 of U.S. Pat. No. 5,817,669, titled “Compounds, processes for the preparation thereof and anti-tumor agents,” issued Oct. 6, 1998, and in Japanese Patent Application No. Hei 10-173986, to Chikugi et al., which are incorporated herein by reference in their entireties. Certain exemplary pharmaceutical compositions comprising SNS-595 and methods of using the same are described in U.S. Patent Application Pub. Nos. 2005-0203120; 2005-0215583 and 2006-0025437, which are incorporated herein by reference in their entireties.

SNS-595 has activity as an anti-tumor agent and is used in methods for treatment, prevention and/or management of one or more cancers. The types of cancers that can be treated, prevented and/or managed using the compound include, but are not limited to solid tumors and blood born tumors. In certain embodiments, cancer is cancer of organs, blood or vessels, including, but not limited to, cancer of the bladder, bone or blood, brain, breast, cervix, chest, colon, endrometrium, esophagus, eye, head, kidney, liver, lung, mouth, neck, ovaries, pancreas, prostate, rectum, stomach, testis, throat or uterus. In certain embodiments, cancer is cancer of skin tissues. In certain embodiments, the cancer is a hematologic malignancy, such as a leukemia, lymphoma (Non-Hodgkin's Lymphoma), Hodgkin's disease (also called Hodgkin's Lymphoma) or myeloma. In certain embodiments, the leukemia is chronic lymphocytic leukemia, chronic myeloid leukemia, acute lymphocytic leukemia, acute myelogenous leukemia or acute myeloblastic leukemia. In certain embodiments, the cancer comprises solid tumor. In certain embodiments, the cancer can be relapsed, refractory or resistant to conventional therapy. In certain embodiments, the cancer can be metastatic.

In certain embodiments, the SNS-595 drug product comprises SNS-595 and an acid. In certain embodiments, the drug product is an aqueous solution of SNS-595 wherein the pH of the solution is less than about 4. In one embodiment, the pH of the solution is from about 2 to about 4, about 2 to about 3.5 or about 2 to about 3. In another embodiment, the pH of the solution is 2.3-2.7.

The acid for use in the drug product is an organic or inorganic acid. Suitable examples of acids include both organic and inorganic acids such as acetic acid, ascorbic acid, benzene-sulfonic acid, ethanesulfonic acid, glycolic acid, hydrogen chloride, hydrogen bromide, hydroxyethanesulfonic acid, lactic acid, maleic acid, methanesulfonic acid, proprionic acid, succinic acid, sulfeuric acid, trifluoroacetic acid, and toluenesulfonic acid. In one embodiment, the acid is hydrochloric acid, methanesulfonic acid or lactic acid. In a particular embodiment, the acid is methanesulfonic acid.

In another embodiment, the drug product comprising SNS-595 further comprises a tonicity agent. Suitable examples of tonicity agent include amino acids (e.g., alanine and glycine), electrolytes (e.g., sodium chloride and potassium chloride), monosaccharides (e.g., glucose or galactose), disaccharides (e.g., sucrose) and hexahydric alcohols (e.g., mannitol and sorbitol). In one embodiment, the tonicity agent is sodium chloride, glucose, mannitol or sorbitol. In another embodiment, the tonicity agent is a hexahydric alcohol. In a particular embodiment, the tonicity agent is sorbitol.

In one embodiment, the SNS-595 drug product is an aqueous solution comprising 10 mg/mL SNS-595 at a pH of about 2.5. In one embodiment, the SNS-595 drug product is an aqueous solution of 100 mg SNS-595 and 450 mg of sorbitol per 10 mL of the solution, wherein the drug product has a pH of 2.5 adjusted with methanesulfonic acid. In certain embodiments, the drug product is for IV administration.

### 5.3 Photostability of Drug Product

The International Conference of Harmonization (ICH) photostability study guidelines indicate that the intrinsic photostability characteristics of drug substances and products should be evaluated to demonstrate that light exposure does not result in unacceptable change in the drug. The ICH guidelines further indicate that a test article should be exposed to 1.2 million lux hours of white light during a photostability study.

As known to one of skill in the art, direct sunlight exposure for about an hour is equivalent to about 100,000 lux hours of white light. Table 1 provides intensity of white light exposure at various locations inside a room and outdoor.

TABLE 1
Light intensity as a function of room type and location

| | Light Intensity | | | |
|---|---|---|---|---|
| | Foot-Candles | | | |
| Location | (FC) | | Lux | |
| Windowless room with fluorescent | 50-60 | FC | 500-600 | lux |
| lighting | | | | |
| Room with windows and fluorescent | 50-60 | FC | 500-600 | lux |
| lighting: measurement in the center | | | | |
| of the room | | | | |
| Room with windows and fluorescent | 200-250 | FC | 2000-2700 | lux |
| lighting: measurement ~1 foot from the | | | | |
| window but not in direct sunlight | | | | |
| Room with windows and fluorescent | ~4,000 | FC | ~44,000 | lux |
| lighting: measurement in direct sunlight | | | | |
| Direct sunlight (early afternoon) | ~10,000 | FC | ~10,000 | lux |

Photostability studies on SNS-595 drug product indicate that the formulation comprising SNS-595 and acid is light sensitive. In certain embodiments, the drug product is stable for up to 2 hours of continuous exposure to ambient light. In certain embodiments, the un-packaged drug product is stable for up to 24 hours when protected from ambient light by wrapping in an aluminum foil.

In certain embodiments, the drug product is stable up to about 78,000 lux hours exposure to light. As described below, the pharmaceutical dosage forms herein provide the desired protection from light for the SNS-595 drug product at greater than 1.2 million lux hours of white light.

### 5.4 Finished Pharmaceutical Products for SNS-595

In certain embodiments, the light protective finished pharmaceutical dosage forms provided herein are pharmaceutical packages for the SNS-595 drug product. The pharmaceutical packages provided herein comprise a primary container housing SNS-595 within a secondary container. The primary container for use herein can be made from any material known to one of skill in the art, including, but not limited to plastic and glass. In one embodiment, the primary container is an ampoule. In another embodiment, the primary container is a vial. In another embodiment, the vial is a glass vial, for example a clear glass vial or an amber glass vial. The vial may contain a screw cap seal, snap off seal or crimped seal. In one embodiment, the vial is a clear glass vial with a crimped seal.

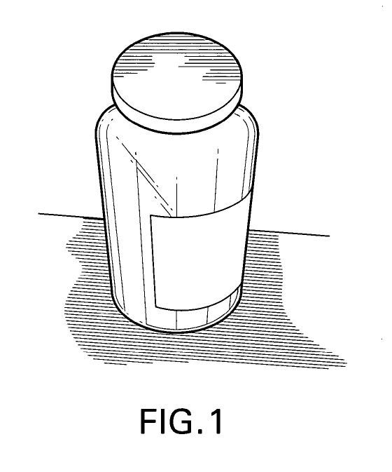

In one embodiment, the vial is a hermetically sealed vial. An exemplary clear glass vial is shown in FIG. 1.

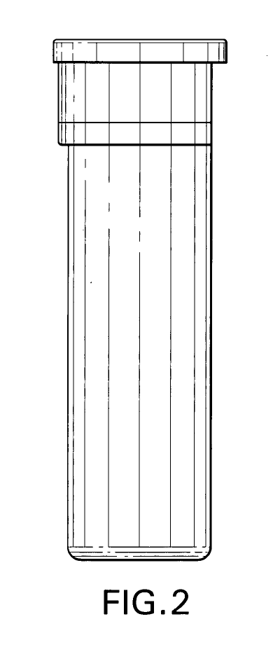

In one embodiment, the light protective pharmaceutical dosage form for SNS-595 drug product comprises an opaque plastic vial with a screw cap, a snap off or a crimped seal. In one embodiment, the vial is hermetically sealed. An exemplary plastic vial is shown in FIG. 2. In certain embodiments, the light protective pharmaceutical packages for a SNS-595 drug product comprise a pre-packed syringe containing SNS-595 within a secondary container. The syringe may be a plastic syringe made from polyethylene, polypropylene, polystyrene or a combination thereof or any other material known to one of skill in the art.

The material used to form the secondary container preferably is economical to procure and can be processed on conventional packaging equipment. The material used to form the secondary container may be also be printable by conventional means on at least one side.

In certain embodiments, the secondary container is a carton constructed of paper or plastic. In certain embodiments, the secondary container is an overwrap or a pouch, which may be fabricated from a wrap composed of a single layer of plastic or a wrap composed of a laminate made of two or more layers selected from the group consisting of metal foil, plastic, and/or paper bonded together by, for example, polyimide wash coatings or primers, various adhesives, and/or thermoplastics. In one embodiment, the secondary container is a box such as a cardboard box that may be a white or brown cardboard box, or is a corrugated box composed of, for example, 1 to 3 layers of paperboard and 1 or 2 layers of flutes, each layer of flutes interspersed between two paperboard layers. The cardboard box may comprise one or more inserts that protect against light. In another embodiment, the secondary container is an opaque, black plastic pouch. In another embodiment, secondary container comprises a plastic film such as polyester and the film is metallized. In another embodiment, the secondary container is an opaque foil-lined pouch.

The pouch can comprise a single sheet of foil that can be folded and sealed all along its edges, or along all non-folded edges. In one embodiment, the pouch comprises two sheets of the foil that can be joined along all its edges. It may further comprise a bag or pocket that can be sealed along one or more edges. The perimeter of the pouch can be in any design, shape or form, irregular or uniform. In certain embodiments, the pouch has a uniform shape such as a square, rectangle, circle or oval in order to facilitate a sealing and/or manufacturing process. In one embodiment, the pouch is rectangular. In another embodiment the pouch has an “H” fold. In another embodiment the “H” fold pouch is a pillow pouch. In another embodiment, the pouch is gusseted and has a “W” fold. In another embodiment the pouch has a “U” fold.

Sealing of the pouch can be accomplished by heat, ultrasound, laser, or adhesive or any other means known to one of skill in the art. In one embodiment, the edges are heat sealed. In one embodiment, the foil is self-sealing (i.e., able to form a stable bond between two facing surfaces of the foil without the use of an adhesive).

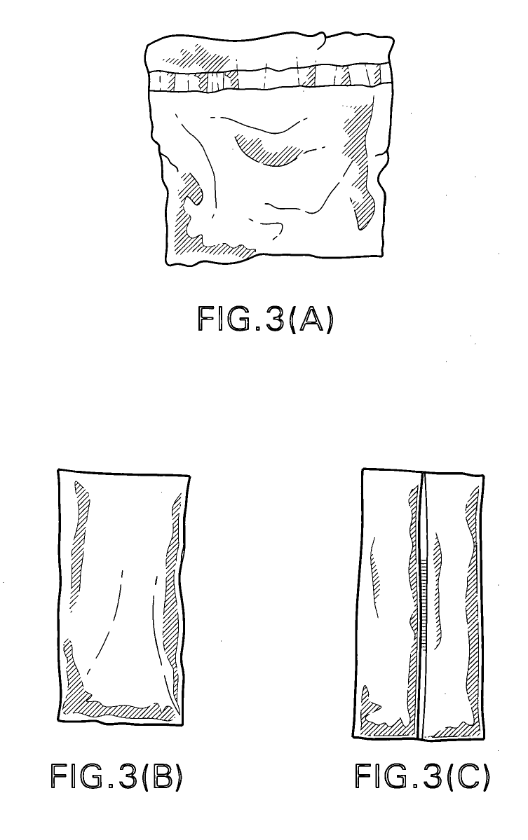

In certain embodiments, the foil-lined pouch comprises an opaque foil. Exemplary foil-lined pouches are shown in FIG. 3(A)-(C). FIG. 3(A) depicts an example of a foil-lined rectangular pouch. FIG. 3(B) and FIG. 3(C) depict the front and back, respectively, of an example of a foil-lined pouch having an “H” fold.

In one embodiment, the foil is a metal foil. The metal may be iron, steel, nickel, copper, tin, bronze, brass, aluminum or other metal known to one of skill in the art.

In one embodiment, the metal is aluminum. In another embodiment, the aluminum comprises alloying elements. Examples of alloys are those having aluminum as the main constituent and the alloying element Fe, Si, Mg, Ti and/or Cu. In one embodiment, the metal foil has a thickness of 0.2 to 200 μm. In another embodiment, the metal foil has a thickness of 0.25 to 200 μm (microns or micrometers). In another embodiment, the metal foil has a thickness of 0.3 to 200 μm. In another embodiment, the metal foil has a thickness of 1 to 200 μm. In another embodiment, the metal foil has a thickness of 5 to 200 μm. In another embodiment, the metal foil has a thickness of 5 to 30 μm. In other embodiments, the metal foil has a thickness of 20 to 200 μm, 25 to 150 μm, 35 to 100 μm, or 40 to 75 μm. In another embodiment, the metal foil has a thickness of 28, 35, 70 or 100 gauge (corresponding to 7 μm, 9 μm, 18 μm, and 25 μm, respectively).

In certain embodiments, the metal foil further comprises a plastic film. The plastic film provides good clarity, may provide good printability and is moisture resistant. The plastic film is chosen such that the plastic used therein can tolerate a wide range of temperatures in the sealing process. The plastic film can comprise polyvinylchloride, polyolefin, polyamide or polyester. In certain embodiments, the plastic film has a thickness of 10 to 250 μm, 10 to 100 μm, or 10 to 50 μm.

In certain embodiments, the plastic film comprises one or more polyolefins, such as polypropylene, low density polypropylene, polyethylene and low density polyethylene. In certain embodiments, useful plastics include those based on halogen-containing polymers, such as polymers of vinylchloride (PVC) and vinyl plastics, containing vinylchloride units in their structure, such as copolymers of vinylchloride and vinylesters of aliphatic acids, copolymers of vinylchloride and esters of acrylic or methacrylic acids or acrylnitrile, copolymers of diene compounds and unsaturated dicarboxyl acids or their anhydrides, copolymers of vinylchloride and vinylchloride with unsaturated aldehydes, ketones etc. or polymers and copolymers of vinylidenchloride with vinylchloride or other polymerizable compounds. The vinyl-based thermoplastics may also be made soft in a conventional manner by means of primary softeners or secondary softeners.

In one embodiment, the material used to prepare the foil-lined pouch comprises polyethylene and foil. In another embodiment, the material used to prepare the foil-lined pouch comprises low density polyethylene and foil. In another embodiment, the material used to prepare the foil-lined pouch comprises polypropylene and foil. In another embodiment, the material used to prepare the foil-lined pouch comprises low density polypropylene and foil.

In another embodiment, the material used to prepare the foil-lined pouch comprises oriented polypropylene, polyethylene, foil and low density polyethylene. In one embodiment, the material used to prepare the foil-lined pouch comprises 90 gauge oriented polypropylene, 15 pound polyethylene, 0.000285 inch thick foil and 40 pound low density polyethylene.

In certain embodiments, the material used to prepare the foil-lined pouch comprises paper, low density polyethylene, and foil. In one embodiment the paper has a basis weight ranging from 12 pound to 60 pound. In another embodiment, the low density polyethylene has a coating weight ranging from 5 pound to 40 pound. In another embodiment, the low density polyethylene has a coating weight ranging from 5 pound to 28 pound. In another embodiment, the foil ranges from 0.00028 inches thick to 0.001 inches thick.

In certain embodiments, the material used to prepare the foil-lined pouch comprises paper, a first layer of low density polyethylene, foil, and a second layer of low density polyethylene. In certain embodiments, the paper is coated on one side only. In certain embodiments the material has an overall thickness of about 2 mils to about 7 mils. In certain embodiments, the material has an overall thickness of about 3 mils to about 5 mils. In certain embodiments, the material has an overall thickness of about 4 mils. As understood by one of skill in the art, the actual material used may have a thickness tolerance of +/−10% of nominal, or more usually, +/−5% of nominal. In certain embodiments, the material used to prepare the foil-lined pouch comprises paper, a first layer of low density polyethylene, foil, and a second layer of low density polyethylene, wherein the first layer of low density polyethylene has a lower coating weight than the second layer of low density polyethylene. In another embodiment, the material used to prepare the foil-lined pouch comprises 26 pound paper, 7.5 pound low density polyethylene, 0.00035 inch foil, and 14.4 pound low density polyethylene. Advantages of the material are, for example, that the outer layer of paper provides mechanical strength and a printable surface, the first layer of LDPE provides the bond between paper and the foil, the foil provides a barrier to light, gas and moisture, and the inner layer of LDPE provides additional strength and enables the material to be heat-sealed.

In one embodiment, the pouch is a rectangular foil-lined pouch that is heat sealed on three sides and comprises oriented polypropylene, polyethylene, foil and low density polyethylene.

In one embodiment, the pharmaceutical package provided herein comprises a clear glass vial with a crimped seal within a foil-lined pouch, wherein the pouch is a rectangular pouch that is heat sealed on three sides and comprises oriented polypropylene, polyethylene, foil and low density polyethylene.

In one embodiment, the pharmaceutical package provided herein comprises a clear glass vial with a crimped seal or a snap off seal within a sealed, opaque, black plastic bag.

In one embodiment, the pouch is a foil-lined pouch that is heat sealed on three sides, has an “H” fold, and comprises paper, a first layer of low density polyethylene, foil, and a second layer of low density polyethylene.

In one embodiment, the pharmaceutical package provided herein comprises a clear glass vial within a foil-lined pouch, wherein the pouch has an “H” fold, is heat sealed on three sides and comprises paper, a first layer of low density polyethylene, foil, and a second layer of low density polyethylene. In one embodiment, the pharmaceutical package provided herein comprises a clear glass vial with a crimped seal within a foil-lined pouch, wherein the pouch is has an “H” fold, is heat sealed on three sides and comprises paper, a first layer of low density polyethylene, foil, and a second layer of low density polyethylene. In another embodiment, the pharmaceutical package provided herein comprises a clear glass vial with a snap off seal within a foil-lined pouch, wherein the pouch has an “H” fold, is heat sealed on three sides and comprises paper, a first layer of low density polyethylene, foil, and a second layer of low density polyethylene.

In one embodiment, the pharmaceutical package provided herein comprises an amber glass within a foil-lined pouch, wherein the pouch has an “H” fold, is heat sealed on three sides and comprises paper, a first layer of low density polyethylene, foil, and a second layer of low density polyethylene. In another embodiment, the pharmaceutical package provided herein comprises an amber glass vial with a crimped seal within a foil-lined pouch, wherein the pouch has an “H” fold, is heat sealed on three sides and comprises paper, a first layer of low density polyethylene, foil, and a second layer of low density polyethylene. In another embodiment, the pharmaceutical package provided herein comprises an amber glass vial with a snap off seal within a foil-lined pouch, wherein the pouch has an “H” fold, is heat sealed on three sides and comprises paper, a first layer of low density polyethylene, foil, and a second layer of low density polyethylene.

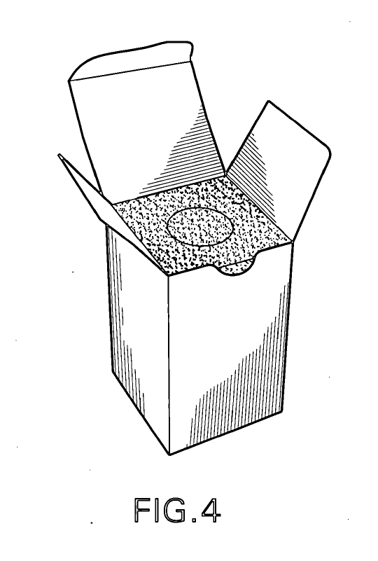

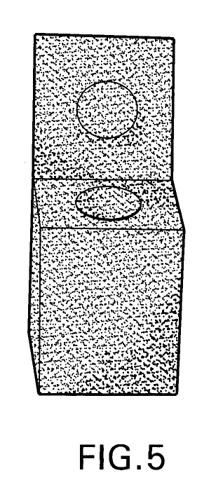

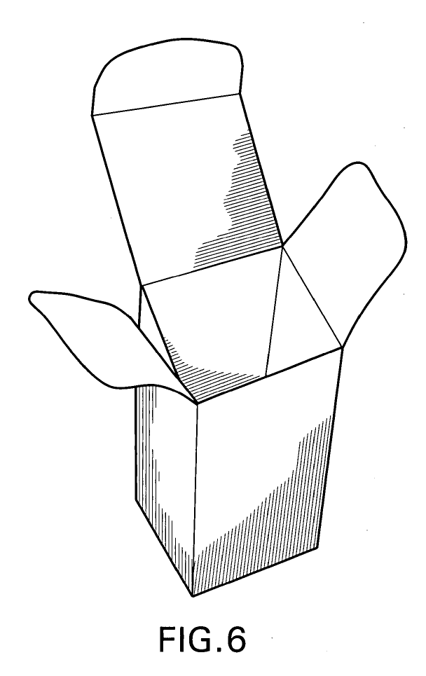

In one embodiment, the secondary container for the pharmaceutical package provided herein comprises a cardboard or a corrugated box. Exemplary white cardboard box is shown in FIG. 4 and a brown corrugated box is shown in FIG. 6. The box may further comprise brown cardboard inserts, folded cardboard inserts or foam lined inserts. In one embodiment, the inserts are black foam lined inserts, for example, as shown in FIG. 5. In one embodiment, the pharmaceutical package provided herein comprises a glass vial within a white cardboard box with black foam lined inserts as shown in FIG. 4.

In certain embodiments, the pharmaceutical package comprises a clear glass vial with a crimped seal or a snap off seal within white cardboard box with black foam lined inserts.

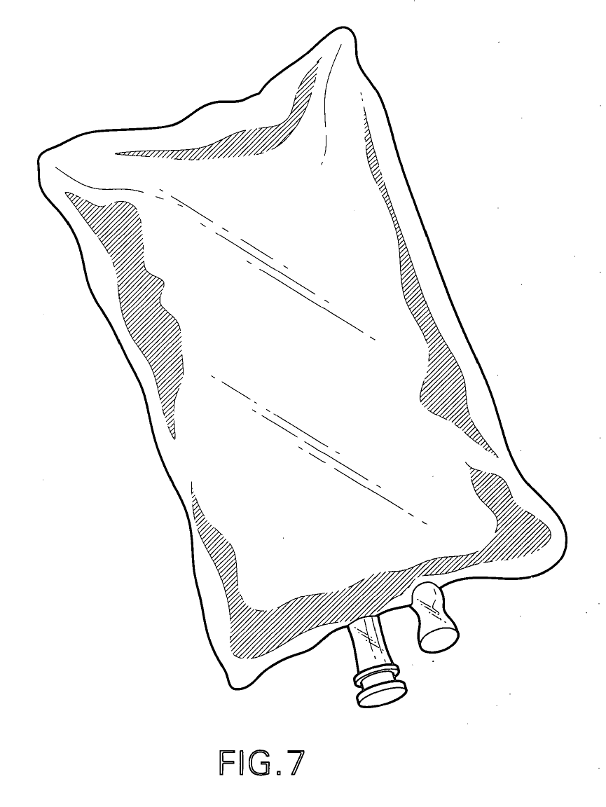

In certain embodiments, the pharmaceutical package comprises an IV bag, wherein the IV bag comprises an opaque foil-lined pouch containing the drug product for IV administration. An exemplary IV bag is shown in FIG. 7.

In certain embodiments, the pharmaceutical package comprises a single light-protective container. For instance, in certain embodiments, the pharmaceutical package can comprise a light-protective vial. In certain embodiments, the pharmaceutical package can comprise a light-protective opaque plastic vial with a screw cap, a snap off or a crimped seal.

In certain embodiments, the pharmaceutical package provided herein allows about 3.5 to about 5.5 lux or about 4 to about 5 lux light penetration. In one embodiment, the pharmaceutical package provided herein allows about 3.5, 4, 4.2, 4.4, 4.6, 4.8 or 5 lux light penetration. In one embodiment, the pharmaceutical package provided herein allows less than about 4.4 lux or about 4.4 lux light penetration.

In certain embodiments, the drug product stored in the pharmaceutical packages provided herein has a shelf-life of from about 1 up to about 36 months. In certain embodiments, the shelf-life is about 2, 4, 6, 8, 10, 12, 14, 16, 18, 20, 22, 24, 30, 36, or 48 months.

The pharmaceutical packages provided herein can comprise unit-dosage or multi-dosage forms of the SNS-595 drug product. In certain embodiments, the pharmaceutical packages comprise from about 1 mg up to about 3000 mg of SNS-595. In certain embodiments, the amount of SNS-595 in the pharmaceutical packages provided herein is about 1-2500 mg, 1-2000 mg, about 1-1500 mg, 1-1300 mg, 1-1200, 1-1000 mg, 1-800 mg, 1-600 mg, 1-500 mg, 1-400 mg, 1-300 mg, 1-200 mg, 1-100 mg, 1-70 mg, 1-50 mg, 1-40 mg, 1-30 mg, 1-25 mg; 1-20 mg, 1-15 mg, 1-10 mg or 1-5 mg.

In certain embodiments, the pharmaceutical packages comprise from about 0.5 mL up to about 300 mL SNS-595 drug product. In certain embodiments, the pharmaceutical packages comprise about 1-250 mL, 1-200 mL, 1-100 mL, 1-50 mL, 1-25 mL, 1-20 mL, 1-10 mL, 1-8 mL, 1-5 mL, 1-4 mL or 1-3 mL SNS-595 drug product. In certain embodiments, the pharmaceutical packages comprise about 1 mL, 2 mL, 3 mL, 4 mL, 5 mL, 6 mL, 7 mL, 8 mL, 9 mL, 10 mL, 12 mL, 15 mL or 20 mL SNS-595 drug product.

### 6. EXAMPLES

Provided herein are non-limiting examples of the claimed subject matter.

### Example 1

### Pharmaceutical Composition Suitable for Injection or Intravenous Infusion

An illustrative example of a suitable composition comprises: 10 mg SNS-595 per mL of aqueous solution of 4.5% sorbitol that is adjusted to pH 2.5 with methanesulfonic acid. One protocol for making such a solution includes the following for making a 100 mg/10 mL formulation: 100 mg of SNS-595 and 450 mg D-sorbitol are added to distilled water; the volume is brought up to a volume of 10 mL; and the pH of the resulting solution is adjusted to 2.5 with methanesulfonic acid. The resulting composition is also suitable for lyophilization. The lyophilized form is then reconstituted with sterile water to the appropriate concentration prior to use.

### Example 2

### Syringe and Infusion Set Compatibility Study

Two BD polypropylene syringes and two Baxter pvc infusion sets were filled 10 mg/mL SNS-595 drug product as prepared above. One syringe and one infusion set were protected from light by wrapping with an aluminum foil. All four items were then exposed to ambient light in a laboratory setting (50-60 foot-candles/500-700 lux) and examined at 1, 2, 4, 8 and 24 hours. Analytical testing at each time point consists of assay/related substances by HPLC. Tables 2-5 provide data for stability of the drug product.

TABLE 2
Polypropylene BD syringe, (wrapped in foil)

| | Approx. | | | | |
|---|---|---|---|---|---|
| Time | Total Lux | Assay | % of | % Impurity | % Total |
| (hours) | Hours | (mg/mL) | Initial | (RRT) | Impurity |
| 0 | 0 | 10.0 | N/A | 0.05 (0.86) | 0.05 |
| 1 | 500-700 | 10.0 | 100.1 | 0.04 (0.86) | 0.04 |
| 2 | 1000-1400 | 10.0 | 99.1 | 0.04 (0.86) | 0.04 |
| 4 | 2000-2800 | 10.0 | 99.7 | 0.04 (0.85) | 0.04 |
| 8 | 4000-5600 | 9.9 | 99.3 | 0.04 (0.85) | 0.05 |
| 24 | 12,000-16,800 | 10.0 | 100.0 | 0.04 (0.86) | 0.05 |

TABLE 3
Polypropylene BD syringe, exposed to ambient light (50-60 foot-
candles/500-700 lux)

| | Approx. | | | | |
|---|---|---|---|---|---|
| Time | Total Lux | Assay | % of | % Impurity | % Total |
| (hours) | Hours | (mg/mL) | Initial | (RRT) | Impurity |
| 0 | 0 | 10.0 | N/A | 0.05 (0.85) | 0.05 |
| 1 | 500-700 | 10.0 | 99.6 | 0.04 (0.85) | 0.04 |
| 2 | 1000-1400 | 10.0 | 99.8 | 0.04 (0.86) | 0.04 |
| 4 | 2000-2800 | 10.0 | 99.5 | 0.04 (0.86) | 0.04 |
| 8 | 4000-5600 | 9.9 | 99.1 | 0.05 (0.86) | 0.05 |
| 24 | 12,000-16,800 | 9.9 | 99.3 | 0.05 (0.85) | 0.05 |

TABLE 4
Baxter PVC infusion set, (wrapped in foil)

| | Approx. | | | | |
|---|---|---|---|---|---|
| Time | Total Lux | Assay | % of | % Impurity | % Total |
| (hours) | Hours | (mg/mL) | Initial | (RRT) | Impurity |
| 0 | 0 | 10.0 | N/A | 0.05 (0.86) | 0.05 |
| 1 | 500-700 | 10.0 | 99.9 | 0.04 (0.85) | 0.04 |
| 2 | 1000-1400 | 10.0 | 99.8 | 0.04 (0.86) | 0.04 |
| 4 | 2000-2800 | 10.0 | 100.0 | 0.04 (0.86) | 0.04 |
| 8 | 4000-5600 | 10.0 | 99.8 | 0.04 (0.86) | 0.04 |
| 24 | 12,000-16,800 | 10.0 | 100.2 | 0.04 (0.86) | 0.04 |

TABLE 5
Baxter PVC infusion set, exposed to ambient light
(50-60 foot-candles/500-700 lux)

| | Approx. | | | | |
|---|---|---|---|---|---|
| Time | Total Lux | Assay | % of | % Impurity | % Total |
| (hours) | Hours | (mg/mL) | Initial | (RRT) | Impurity |
| 0 | 0 | 10.0 | N/A | 0.04 (0.86) | 0.05 |
| 1 | 500-700 | 10.0 | 99.6 | 0.05 (0.85) | 0.04 |
| 2 | 1000-1400 | 10.0 | 99.7 | 0.04 (0.86) | 0.04 |
| 4 | 2000-2800 | 9.9 | 99.2 | 0.04 (0.86) | 0.04 |
| 8 | 4000-5600 | 9.9 | 99.2 | 0.03 (0.33), | 0.08 |
| | | | | 0.05 (0.85) | |
| 24 | 12,000-16,800 | 10.0 | 100.0 | 0.03 (0.73), | 0.13 |
| | | | | 0.03 (0.82), | |
| | | | | 0.07 (0.85) | |

The SNS-595 drug product in aluminum foil covered syringe and/or infusion set exposed to ambient light condition was stable up to 24 hours, at which point an Assay and Related Substances analysis of the drug product showed it was unchanged from its T=0 condition (see Tables 2 and 4 at the 24 hour time-points).

### Example 3

### ICH Photostability Study

SNS-595 Drug Product (10 mg/mL) in USP Type I clear glass vial from Schott/West (Product #680000320) with a stopper (American Stelmi, Product CIC1474 6720 GC6TP) was exposed to visible light per the International Conference of Harmonization (ICH) guidelines and examined at 1, 2, 4, 8 and 24 hours. Analytical testing at each time point includes appearance, pH, and assay/related substances by HPLC.

TABLE 6
ICH photostability Study

| | | | Assay | | |
|---|---|---|---|---|---|
| Time | Total Lux | | % SNS- | Rel. Substances | |
| (hours) | Hours¹ | Appearance | 595 | % Impurity (RRT) | pH |
| 0 | 0 | Clear, colorless | 99.0 | Not Reported | 2.5 |
| | | liquid | | | |
| 1 | 9791.6 | Clear, colorless | 99.0 | 0.1% (0.85) | 2.5 |
| | | liquid | | Total: 0.1% | |
| 2 | 19583.2 | Clear, colorless | 98.1 | 0.1% (0.66), 0.1% | 2.5 |
| | | liquid | | (0.78), 0.2% (0.85); | |
| | | | | Total: 0.4% | |
| 4 | 39166.4 | Clear, colorless | 96.3 | 0.2% (0.66), 0.3% | 2.5 |
| | | liquid | | (0.78), 0.3% (0.85); | |
| | | | | Total: 0.8% | |
| 8 | 78332.8 | Clear, colorless | 95.4 | 0.1% (0.48), 0.2% | 2.5 |
| | | liquid | | (0.66), 0.5% (0.78), | |
| | | | | 0.4% (0.85), 0.1% | |
| | | | | (1.43); | |
| | | | | Total: 1.3 | |
| 24 | 234988.4 | Clear, colorless | 90.7 | 0.2% (0.48), 0.1% | 2.5 |
| | | liquid | | (0.50), 0.1% (0.63), | |
| | | | | 0.6% (0.66), 0.1% | |
| | | | | (0.67, 0.1% (0.73), | |
| | | | | 1.3% (0.78), 0.2% | |
| | | | | (0.82), 0.8% (0.85), | |
| | | | | 0.2% (1.37), 0.2% | |
| | | | | (1.43), 0.1% (1.58); | |
| | | | | Total: 4.1% | |

¹ICH conditions require exposure to 1.2 million lux hours of light, but the study was terminated after only 234,000 lux hours due to extensive degradation of the sample.

The data indicated extensive degradation of SNS-595 in the drug product after only 234,000 lux hours exposure to light. Therefore, the study was terminated after 234,000 lux hours.

### Example 4

### Photostability Study in Amber Glass Vial

SNS-595 Drug Product (10 mg/mL) was packaged in amber vials. Samples were exposed to visible light per the International Conference of Harmonization (ICH) guidelines and examined at 4, 8, 24.5, 48.5, 97, 115 and 321 hours. Table 7 provides % area for SNS-595 and total impurities assayed by HPLC at each time point.

TABLE 7
Amber glass vial exposed to white light

| | | | Total |
|---|---|---|---|
| Time | Total Lux | SNS-595 | Impurities |
| (hours) | Hours | (% Area) | (% Area) |
| 0 | 0 | 99.4 | 0.4 |
| 4 | ~50000 | 99.6 | 0.2 |
| 82 | ~100000 | 99.6 | 0.3 |
| 24.5 | ~300000 | 99.6 | 0.2 |
| 48.5 | ~600000 | 99.5 | 0.1 |
| 97 | ~1000000 | 99.1 | 0.6 |
| 115 | ~1400000 | 98.9 | 0.9 |

As seen from the data presented in Table 7, exposure of the SNS-595 drug product in amber vial to about 1 million lux hours of light resulted in formation of degradation products at longer exposure times.

### Example 5

### Photostability Study in Clear Glass Vial in a Cardboard Box

SNS-595 Drug Product (10 mg/mL) is packaged in clear glass vials. One vial is protected from light by enclosing in a cardboard box. Samples are exposed to visible light per the International Conference of Harmonization (ICH) guidelines and examined at about 4, 8, 24.5, 48.5, 97, 115 and 321 hours.

### Example 6

### Photostability Study in Clear Glass Vial with and without Aluminum Foil Wrap

SNS-595 Drug Product (10 mg/mL) was packaged in clear glass vials. One vial was protected from light by wrapping with aluminum foil. Both the samples were exposed to visible light per the International Conference of Harmonization (ICH) guidelines and examined at 4, 8, 24, 48, 96, and 120 hours. Analytical testing at each time point consisted of assay/related substances by HPLC.

TABLE 8
Clear glass vial, exposed

| | | Assay | |
|---|---|---|---|
| Time | Total Lux | % SNS- | Rel. Substances |
| (hours) | Hours¹ | 595 | % Impurity (RRT)¹ |
| 0 | 0 | 99.7 | Not Reported |
| 4 | 49488 | 99.2 | 0.1 (0.33), 0.2 (0.86), 0.2 (1.84), |
| | | | 0.1 (2.15); |
| | | | Total: 0.6% |
| 8 | 98976 | 98.1 | Not Reported |
| 24.5 | 306299 | 94.4 | Not Reported |
| 48.5 | 608028 | 91.5 | Not Reported |
| 97 | 1223170 | 87.6 | Not Reported |
| 115 | 1458449 | 88.0 | 28 impurities ranging from 0.1-2.4%, |
| | | | (0.33-2.17); |
| | | | Total: 13.0% |

¹Impurities present at ≧0.05% reported. Impurities present at <0.05% omitted.

TABLE 9
Clear glass vial, wrapped in foil

| Time | Total Lux | Assay | Rel. Substances |
|---|---|---|---|
| (hours) | Hours¹ | % SNS-595 | % Impurity (RRT) |
| 0 | 0 | 99.7 | Not Reported |
| 4 | 49488 | 99.8 | Not Reported |
| 82 | 98976 | 99.8 | Not Reported |
| 24.5 | 306299 | 99.8 | Not Reported |
| 48.5 | 608028 | 99.8 | Not Reported |
| 97 | 1223170 | 99.8 | Not Reported |
| 115 | 1458449 | 99.7 | Not Reported |

¹Impurities present at ≧0.05% reported. Impurities present at <0.05% omitted

Analysis of the drug product in a clear glass vial indicated that exposure to about 1.4 million lux hours resulted in degradation of about 11.7% SNS-595 in the drug product. The sample in a clear glass vial covered with aluminum foil exposed over 1.4 million lux hours of white light showed no change in assay (see Table 9).

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1-38. (canceled)

39. A pharmaceutical dosage form of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid comprising a primary container containing a drug product comprising (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, wherein the primary container is within an opaque secondary container.

40. The pharmaceutical dosage form of claim 1, wherein the primary container is a glass vial.

41. The pharmaceutical dosage form of claim 2, wherein the primary container is a clear glass vial or an amber glass vial.

42. The pharmaceutical dosage form of claim 2, wherein the secondary container is a foil-lined pouch.

43. The pharmaceutical dosage form of claim 4, wherein the foil consists essentially of aluminum.

44. The pharmaceutical dosage form of claim 5, wherein the foil has a thickness of 0.3 to 200 μm.

45. The pharmaceutical dosage form of claim 6, wherein the foil has a thickness of 5 to 200 μm.

46. The pharmaceutical dosage form of claim 7, wherein the foil has a thickness of 5 to 30 μm.

47. The pharmaceutical dosage form of claim 1, wherein the drug product further comprises an acid selected from the group consisting of acetic acid, ascorbic acid, benzenesulfonic acid, ethanesulfonic acid, glycolic acid, hydrogen chloride, hydrogen bromide, hydroxyethanesulfonic acid, lactic acid, maleic acid, methanesulfonic acid, propionic acid, succinic acid, sulfuric acid, trifluoroacetic acid, and toluenesulfonic acid.

48. The pharmaceutical dosage form of claim 9, wherein the acid is methanesulfonic acid.

49. The pharmaceutical dosage form of claim 1, wherein the drug product further comprises water.

50. The pharmaceutical dosage form of claim 11, wherein the drug product has a pH of less than 4.

51. The pharmaceutical dosage form of claim 12, wherein the drug product has a pH of from about 2.3 to about 2.7.

52. The pharmaceutical dosage form of claim 13, wherein the drug product has a pH of about 2.5.

53. The pharmaceutical dosage form of claim 1, further comprising a tonicity agent selected from the group consisting of amino acids, electrolytes, monosaccharides, disaccharides, and hexahydric alcohols.

54. The pharmaceutical dosage form of claim 15, wherein the tonicity agent is sorbitol.

55. The pharmaceutical dosage form of claim 1, wherein the drug product is an aqueous solution comprising 10 mg/mL of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

56. The pharmaceutical dosage form of claim 1, wherein the drug product is an aqueous solution comprising 4.5% sorbitol.

57. The pharmaceutical dosage form of claim 1, wherein the drug product is an aqueous solution comprising 100 mg of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid and 450 mg of sorbitol per 10 mL.

58. The pharmaceutical dosage form of claim 19, wherein the drug product comprises 100 mg of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid and 450 mg of sorbitol per 10 mL of the aqueous solution, and sufficient methanesulfonic acid to provide the aqueous solution a pH of about 2.5.

59. The pharmaceutical dosage form of claim 13 wherein the drug product is aqueous and the primary container contains from about 1 mL to about 200 mL of the drug product.

60. The pharmaceutical dosage form of claim 21, wherein the primary container contains from about 1 mL to about 20 mL of the drug product.

61. The pharmaceutical dosage form of claim 22, wherein the primary container contains from about 1 mL to about 10 mL of the drug product.

62. The pharmaceutical dosage form of claim 23, wherein the primary container contains from about 1 mL to about 5 mL of the drug product.

63. The pharmaceutical dosage form of claim 1, wherein the primary container contains from about 1 mg to about 200 mg of the (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid.

64. A pharmaceutical dosage form of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid drug product, wherein the dosage form allows less than about 6 lux light penetration under ambient light.

65. A pharmaceutical dosage form of (L)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, comprising a glass vial containing a drug product comprising (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, wherein the glass container is within an opaque overwrap.

66. A pharmaceutical dosage for of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, comprising a glass vial containing a drug product comprising (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, wherein the glass container is within a second container that allows less than about 6 lux penetration under ambient light.

67. A pharmaceutical dosage form of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, comprising a glass vial containing a drug product comprising (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid; wherein the glass container is within a second container, whereby the drug product is photostable for up to 48 months under ambient light.

68. A pharmaceutical dosage for of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, comprising a glass vial containing a drug product comprising (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid; wherein the glass container is within a second container, whereby the drug product is photostable at exposure of up to 1.2 million lux hours of white light.

69. A pharmaceutical dosage form of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, comprising a container containing from about 1 mL to about 50 mL of an aqueous solution comprising 10 mg/mL of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, 45 mg/mL of sorbitol, and sufficient methanesulfonic acid to adjust the pH of the drug product to about 2.5; wherein the container allows less than about 6 lux penetration under ambient light.

70. A pharmaceutical dosage form of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, comprising a clear glass vial within a pouch, wherein the glass vial contains from about 1 mL to about 50 mL of aqueous solution comprising 10 mg/mL of (+)-1,4-dihydro-7-[(3S,4S)-3-methoxy-4-(methylamino)-1-pyrrolidinyl]-4-oxo-1-(2-thiazolyl)-1,8-naphthyridine-3-carboxylic acid, 45 mg/mL of sorbitol, and sufficient methanesulfonic acid to adjust the pH of the drug product to about 2.5; and wherein the pouch allows less than about 6 lux penetration under ambient light.
